(19) United States
(12) Patent Application Publication
(10) Pub. No.: US 2023/0268077 A1
(43) Pub. Date: Aug. 24, 2023

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventor: Ryo HIRANO, Otawara (JP)

(73) Assignee: CANON MEDICAL SYSTEMS CORPORATION, Otawara-shi (JP)

(21) Appl. No.: 18/166,001

(22) Filed: Feb. 8, 2023

(30) Foreign Application Priority Data
Feb. 18, 2022 (JP) 2022-023821

(51) Int. Cl. G16H 50/30; G16H 20/00; G16H 10/60

(52) U.S. Cl. CPC G16H 50/30; G16H 20/00; G16H 10/60

(57) ABSTRACT

A medical information processing device according to an embodiment includes processing circuitry. The processing circuitry is configured to acquire first risk information regarding a first disease of a subject, and acquire second risk information regarding a second disease different from the first disease when the acquired first risk information satisfies a condition based on a second threshold value which is less than a first threshold value related to treatment determination of the first disease and is greater than a normal range.

### CROSS-REFERENCE TO RELATED APPLICATION

The present application claims priority based on Japanese Patent Application No. 2022-023821 filed Feb. 18, 2022, the content of which is incorporated herein by reference.

### FIELD

The embodiments disclosed in this specification and drawings relate to a medical information processing device, a medical information processing method, and a storage medium.

### BACKGROUND

In recent years, technology for automatically diagnosing specific diseases, which are examination targets, by analyzing medical data such as medical image data and vital data has become known. Further, technology for presenting diagnostic results regarding diseases other than examination targets by using supplementary information (patient information and the like) of medical data acquired for diagnosing diseases, which are examination targets, has also been proposed.

When automatic diagnosis is applied to all medical data in a situation in which the number of normal cases is extremely large compared to the number of abnormal cases, the number of cases (false positive results) in which a normal case is diagnosed as an abnormal case may be an unacceptable number. As a result, the reliability of automatic diagnosis decreases, and situations in which doctors and patients cannot trust diagnosis results occur. In order to avoid such a situation, it is desirable to narrow down the number of patients who are targets for automatic diagnosis.

In addition, when automatic diagnosis of a disease, which is not an examination target, is performed using supplementary information of medical data, the risk of being affected by each disease is not necessarily reflected in the supplementary information. For this reason, this conventional method does not necessarily lead to narrowing down the number of patients who are targets for automatic diagnosis to a high-risk group for the corresponding disease, causing the number of false positive results to increase.

### BRIEF DESCRIPTION OF DRAWINGS

FIG. 1 is a diagram showing an example of a usage environment and functional blocks of a medical information processing device 1 according to a first embodiment.

FIG. 2 is a diagram illustrating an example of a case in which the medical information processing device 1 according to the first embodiment is applied to a diagnosis flow.

FIG. 3 is a flowchart showing an example of a flow of processing of the medical information processing device 1 according to the first embodiment.

FIG. 4 is a diagram illustrating an example when a medical information processing device 1 according to a second embodiment is applied to a diagnosis flow.

FIG. 5 is a flowchart showing an example of a flow of processing of the medical information processing device 1 according to the second embodiment.

FIG. 6 is a diagram showing an example of a usage environment and functional blocks of a terminal device 5 according to a modified example.

### DETAILED DESCRIPTION

A medical information processing device, a medical information processing method, and a storage medium according to embodiments will be described below with reference to the drawings. A medical information processing device according to an embodiment acquires first risk information regarding a first disease of a patient (subject), and when the acquired first risk information satisfies a predetermined condition, acquires second risk information regarding a second disease that is different from the first disease of the subject. In the present embodiment, diseases refer to specific diseases such as diabetes, liver fibrosis, liver cirrhosis, cancer, myocardial infarction, and stroke. In addition, these diseases may include pre-diseases in which there is not a healthy state but which have not yet developed in addition to diseases that have developed. Risk information refers to information related to a disease, such as the presence or absence of the disease, the severity of the disease, and the time of onset of the disease. Risk information includes measurement data itself measured using a measurement device, index values calculated on the basis of the measurement data, diagnosis results based on the measurement data, and the like.

In embodiments, the first disease and the second disease have a predetermined relationship. For example, the second disease is a disease that inhibits treatment of the first disease. For example, the second disease is liver dysfunction (liver fibrosis, liver cirrhosis, and the like) and the first disease is diabetes. Some treatment methods for diabetes (for example, methods of treatment using tolbutamide) are known to impair liver function. Therefore, at the time of treating diabetes (second disease), treatment methods that may reduce liver function cannot be applied if the patient suffers from liver dysfunction (first disease). In this manner, the first disease and the second disease have a relationship regarding treatment methods, for example.

The medical information processing device can narrow down the number of targets for acquisition of the second risk information by being configured to acquire the second risk information only when the first risk information satisfies a predetermined condition. Accordingly, it is possible to reduce the number of false positive results. In addition, it is possible to increase the likelihood of being able to apply an appropriate treatment method to the first disease by treating the second disease at a stage before the first disease worsens and requires treatment.

### First Embodiment

### Configuration of Medical Information Processing Device

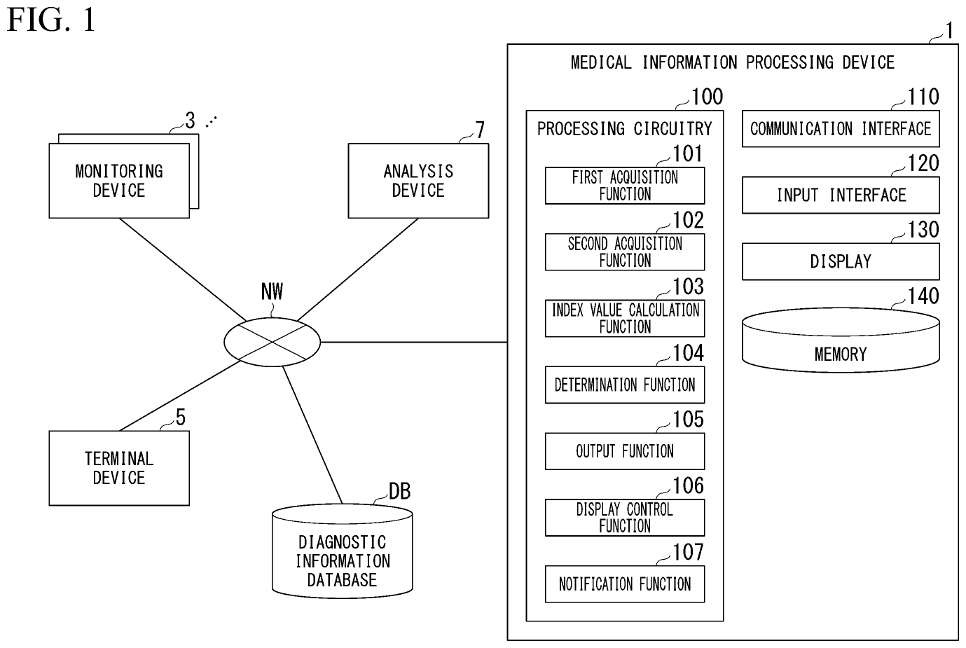

FIG. 1 is a diagram showing an example of a usage environment and functional blocks of a medical information processing device 1 according to a first embodiment. The medical information processing device 1 is installed, for example, in a medical institution such as a hospital. The medical information processing device 1 is operated by an operator such as a doctor, for example. The medical information processing device 1 may be, for example, a workstation, a server, a console device of a medical image diagnostic apparatus, or the like. For example, the medical information processing device 1 is connected to at least one monitoring device 3, terminal device 5, analysis device 7, diagnostic information database DB, and the like via a communication network NW such that data can be transmitted and received therebetween. One of the medical information processing device 1 and the terminal device 5 or a combination of both is an example of a “medical information processing device.”

The communication network NW indicates a general information communication network using telecommunication technology. The communication network NW includes a telephone communication network, an optical fiber communication network, a cable communication network, a satellite communication network, and the like in addition to a wireless/wired local area network (LAN) such as a hospital backbone LAN, and the Internet.

The monitoring device 3 periodically measures monitoring data related to diseases from patients. The monitoring device 3 is installed, for example, in a medical institution such as a hospital, a patient's home, or the like. The monitoring device 3 may be constantly worn by a patient to perform measurement. For example, the monitoring device 3 periodically measures monitoring data relating to the first disease from the patient. Items of monitoring data include at least one of items that are related to a disease and that are preferably measured periodically to check the status of the disease. For example, if the first disease is diabetes, items of monitoring data include a body weight, a body mass index (BMI), index values related to exercise habits (number of steps, calories burned, etc.), a blood pressure, blood test data, and the like. Monitoring devices include, for example, weighing scales, electrocardiographs, heart rate monitors, pedometers, blood test devices, and the like. The monitoring device 3 may be, for example, a single device such as a smart watch, which has a function of measuring a plurality of items.

The monitoring device 3 transmits measured measurement data (hereinafter referred to as “monitoring data”) to the medical information processing device 1, the terminal device 5, and the like via the communication network NW. The monitoring data is saved in any format in any storage device of each device. The monitoring data includes numerical data, image data, text data, voice data, image data obtained by imaging a patient's own handwriting on paper, and the like.

The frequency of measurement by the monitoring device 3 may be equal to or higher than a frequency recommended for each monitoring item related to the first disease. Further, when there are a plurality of monitoring items related to the first disease, the frequency of measurement of each monitoring item may be different. In this case, interpolation processing or filter processing may be applied to measurement items with a low measurement frequency to match data intervals and the number of pieces of data with those of measurement items with a high measurement frequency. Further, data may be thinned out or filter processing may be applied to measurement items with a high measurement frequency to match data intervals and the number of pieces of data with those of measurement items with a low measurement frequency. In cases in which acquired measured values are greatly different from previous measurement results or are physiologically or physically impossible values, and the like, processing of excluding or correcting measured values that are determined to be abnormal values may be applied. Meanwhile, a method of performing monitoring is not limited. The monitoring device 3 may be given to a patient to allow the patient to perform measurement and recording. Alternatively, a patient may be called to a space provided in a medical institution or a facility corresponding thereto is provided, such as one for regular health checkups, and monitoring data may be measured and recorded by a doctor, a technician, or the like.

The terminal device 5 is, for example, a mobile terminal such as a tablet or a smartphone carried by a patient, a personal computer, or the like. The terminal device 5 is operated by, for example, a patient who is a target for monitoring, a caregiver, or the like. The terminal device 5 stores monitoring data measured by the monitoring device 3 and transmits the monitoring data to the medical information processing device 1. In addition, the terminal device 5 activates a dedicated application program, a browser, or the like and notifies the patient of various types of information provided by the medical information processing device 1.

The analysis device 7 automatically analyzes for a specific disease using diagnostic information and outputs analysis results. The analysis device 7 analyzes for the second disease using, for example, medical image data stored in the diagnostic information database DB. Each function of the analysis device 7 may be incorporated in the medical information processing device 1.

The diagnostic information database DB stores diagnostic information of a plurality of patients. In diagnostic information, for example, patient identification information (patient ID) is associated with medical image data captured in the past, electronic medical records, disease information, patient information such as age and sex, biological information, and the like. Medical image data includes, for example, computed tomography (CT) images, ultrasonic diagnostic images, magnetic resonance (MR) images, X-ray images, and the like. Biological information includes, for example, a blood pressure value, a pulse rate, a respiration rate, and the like. The diagnostic information database DB is realized by, for example, a random access memory (RAM), a semiconductor memory device such as a flash memory, a hard disk, an optical disc, and the like.

The medical information processing device 1 includes, for example, processing circuitry 100, a communication interface 110, an input interface 120, a display 130, and a memory 140. The communication interface 110 communicates with external devices such as the monitoring device 3, the terminal device 5, the analysis device 7, and the diagnostic information database DB via the communication network NW. The communication interface 110 includes, for example, a communication interface such as a network interface card (NIC).

The input interface 120 receives various input operations from the operator of the medical information processing device 1, converts the received input operations into electrical signals, and outputs the electrical signals to the processing circuitry 100. For example, the input interface 120 includes a mouse, a keyboard, a trackball, switches, buttons, a joystick, a touch panel, and the like. The input interface 120 may be, for example, a user interface that receives voice input, such as a microphone.

In this specification, the input interface is not limited to those having physical operation parts such as a mouse and a keyboard. For example, the input interface also includes electrical signal processing circuitry that receives an electrical signal corresponding to an input operation from an external input device provided separately from the apparatus and outputs the electrical signal to a control circuit.

The display 130 displays various types of information. For example, the display 130 displays images generated by the processing circuitry 100, a graphical user interface (GUI) for receiving various input operations from the operator, and the like. For example, the display 130 is a liquid crystal display (LCD), a cathode ray tube (CRT) display, an organic electroluminescence (EL) display, or the like. The display function of the display 130 may be incorporated into the input interface 120 when the input interface 120 is a touch panel.

The processing circuitry 100 includes, for example, a first acquisition function 101, a second acquisition function 102, an index value calculation function 103, a determination function 104, an output function 105, a display control function 106, and a notification function 107. The processing circuitry 100 realizes these functions by, for example, a hardware processor (computer) executing a program stored in the memory 140 (storage circuit).

The hardware processor refers to, for example, a circuitry such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). A configuration in which the program is directly incorporated into the circuitry of the hardware processor instead of being stored in the memory 140 may be adopted. In this case, the hardware processor realizes the functions by reading and executing the program incorporated into the circuitry. The aforementioned program may be stored in the memory 140 in advance, or may be stored in a non-transitory storage medium such as a DVD or CD-ROM and installed in the memory 140 from the non-transitory storage medium when the non-transitory storage medium is set in a drive device (not shown) of the medical information processing device 1. The hardware processor is not limited to being configured as a single circuit, and may be configured as one hardware processor by combining a plurality of independent circuits to realize each function. Further, a plurality of components may be integrated into one hardware processor to realize each function.

The first acquisition function 101 acquires monitoring data regarding the first disease from the monitoring device 3, the terminal device 5, or the diagnostic information database DB via the communication network NW. The first acquisition function 101 also acquires a risk index value (first risk index value) of the first disease output from the index value calculation function 103 and outputs the risk index value to the determination function 104. The first acquisition function 101 may search the memory 140 in which the first risk index value of the patient who is a target for diagnosis is stored and acquire the first risk index value or may acquire that manually input by a doctor, the patient, or a person related thereto via the input interface 120. The first acquisition function 101 may perform filtering, correction, or the like on the first risk index value. For example, it is assumed that calculation of the first risk index value has already been performed multiple times for the patient who is a target for diagnosis and the memory 140 stores change in the first risk index value over time. In this case, the first acquisition function 101 can reduce the influence of deviations caused by measurement errors of the monitoring device 3 and error propagation by applying a moving average filter to the change in the risk index value of the first disease B over time. Monitoring data or the first risk index value is an example of “first risk information.” The first acquisition function 101 is an example of a “first acquirer.” That is, the first acquisition function 101 acquires first risk information regarding the first disease of a subject. The first acquisition function 101 acquires monitoring data obtained by monitoring the subject.

The second acquisition function 102 acquires diagnostic information on the second disease from the diagnostic information database DB via the communication network NW and stores the diagnostic information in the memory 140. The second acquisition function 102 may acquire diagnostic information manually input by a doctor, the patient, or a person related thereto via the input interface 120. The second acquisition function 102 also acquires analysis results based on diagnostic information on the second disease by the analysis device 7 via the network NW when the first risk information satisfies a predetermined condition. The diagnostic information on the second disease or the analysis result based on the diagnostic information on the second disease is an example of “second risk information.” The second acquisition function 102 is an example of a “second acquirer.” That is, the second acquisition function 102 acquires the second risk information regarding the second disease different from the first disease of the subject when the acquired first risk information satisfies a predetermined condition. The second acquisition function 102 acquires the second risk information regarding the second disease different from the first disease when the acquired first risk information satisfies a condition based on a second threshold value which is less than a first threshold value related to treatment determination of the first disease and is greater than a normal range. The second acquisition function 102 acquires the second risk information, which is an analysis result of the second disease, from the analysis device 7. The second acquisition function 102 acquires past diagnostic information on a subject, and acquires second risk information obtained by analyzing the past diagnostic information.

The index value calculation function 103 calculates a first risk index value on the basis of monitoring data regarding the first disease acquired by the first acquisition function 101. The index value calculation function 103 may calculate a plurality of first risk index values. The index value calculation function 103 may be provided in a processing apparatus separate from the medical information processing device 1. Any type of first risk index value may be used as long as it is an index value calculated on the basis of monitoring data regarding the first disease measured by the monitoring device 3. The first risk index value includes, for example, a probability of developing the first disease in the future, severity, urgency, treatment priority, a survival rate within a certain period, an overall survival period, and the like. The index value calculation function 103 calculates the first risk index value using any method. The index value calculation function 103 may receive measured values of a plurality of monitoring items as inputs, for example, and predict a risk index value using regression analysis, a neural network, a decision tree, a naive Bayesian classifier, or the like. Further, the index value calculation function 103 may use only one monitoring item to calculate the first risk index value. For example, if a certain monitoring item is strongly correlated with at least one first risk index value, the index value calculation function 103 may use the measured value of the monitoring item itself as the first risk index value. In addition, the index value calculation function 103 may calculate the first risk index value by performing normalization by multiplying the measured value of the monitoring item by a coefficient, or the like. The first risk index value may be a continuous value. Further, the first risk index value may be a value obtained by classifying the risk index value of the first disease according to size. For example, the first risk index value may be represented by letters indicating a degree of risk such as “low,” “medium,” or “high,” a symbol such as “+,” “−,” “▴” or “▾,” or a discontinuous value. The index value calculation function 103 is an example of an “index value calculator.” That is, the index value calculation function 103 calculates the first risk information, which is the index value related to the first disease, on the basis of the acquired monitoring data.

The determination function 104 determines whether or not to analyze for the second disease on the basis of the first risk index value calculated by the index value calculation function 103. For example, the determination function 104 sets a threshold value for the first risk index value and determined whether or not to analyze for the second disease on the basis of comparison between the first risk index value and the threshold value (e.g., on the basis of whether the first risk index value is equal to or greater than the threshold value or exceeds the threshold value). In addition, when there are a plurality of types of first risk index values, the determination function 104 may determine whether or not to analyze for the second disease using regression analysis, a convolutional neural network, a decision tree, a naive Bayes classifier, or the like. The determination function 104 is an example of a “determiner.” That is, the determination function 104 determines whether or not to analyze for the second disease on the basis of whether the acquired first risk information satisfies a predetermined condition. The determination function 104 determines whether or not to analyze the second disease on the basis of comparison between the first risk information and the predetermined threshold value. The determination function 104 determines to analyze the second disease when the acquired first risk information is equal to or greater than the second threshold value or is greater than the second threshold value.

The output function 105 outputs diagnostic information regarding the second disease acquired by the second acquisition function 102 to the analysis device 7. The analysis device 7 analyzes for the second disease using the diagnostic information acquired from the output function 105 and outputs the analysis result to the medical information processing device 1, the diagnostic information database DB, and the like. The output function 105 is an example of an “output.” That is, when the determination function 104 determines that the second disease will be analyzed, the output function 105 outputs instruction information for instructing analysis of the second disease using the diagnostic information regarding the subject to the external analysis device 7.

The display control function 106 performs control of causing the display 130 to display various types of information such as monitoring data, the first index value, diagnostic information on the second disease, analysis results obtained by the analysis device 7, and a GUI for receiving various input operations from the operator.

The notification function 107 notifies the terminal device 5, a smartwatch, or the like carried by the patient of analysis results regarding the second disease output by the analysis device 7 and instruction information (support information) for instructing treatment for the second disease to be started via the communication network NW. The notification function 107 is an example of a “notifier.” That is, the notification function 107 notifies of support information regarding the first or second disease on the basis of the acquired first or second risk information. The notification function 107 notifies of the support information for instructing treatment for the second disease to be started on the basis of the acquired second risk information.

The memory 140 is realized by, for example, a RAM, a semiconductor memory device such as a flash memory, a hard disk, or an optical disc. These non-transitory storage media may be realized by other storage devices such as a network attached storage (NAS) and an external storage server device connected via the communication network NW. The memory 140 may also include non-transitory storage media such as a read only memory (ROM) and a register. The memory 140 stores, for example, monitoring data, the first index value, diagnostic information on the second disease, analysis results regarding the second disease, and the like. In addition, the memory 140 stores programs, parameter data, and other data used by the processing circuitry 100.

### Processing Flow

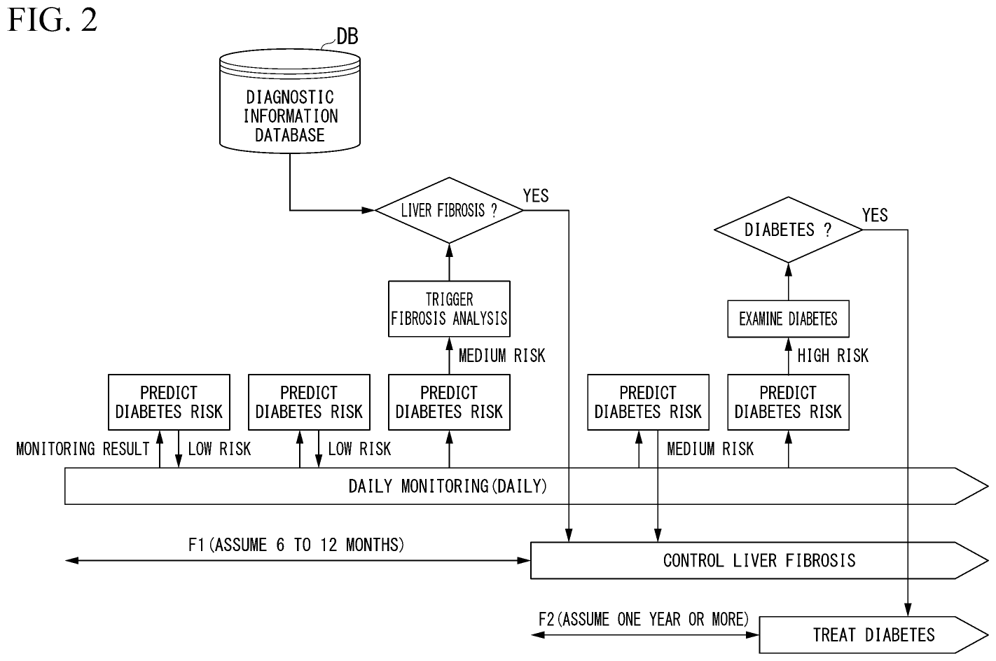

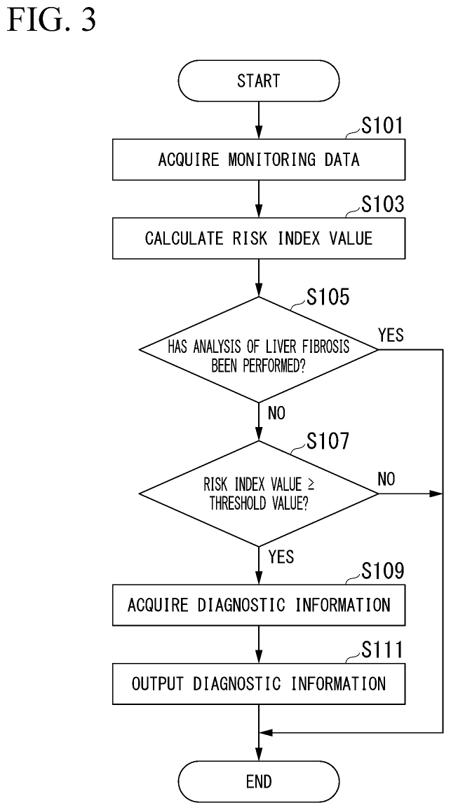

Next, an example of a processing flow of the medical information processing device 1 according to the first embodiment will be described. FIG. 2 is a diagram illustrating an example of a case in which the medical information processing device 1 according to the first embodiment is applied to a diagnosis flow. FIG. 3 is a flowchart showing an example of a flow of processing of the medical information processing device 1 according to the first embodiment. In the following, an example of a case in which the first disease is “diabetes” and the second disease is “liver fibrosis” will be described. In addition, it is assumed that a patient who is a target for diagnosis has undergone a CT examination of an organ around the liver (for example, examination of pneumonia) within the past year and CT images at that time are stored in the diagnostic information database DB. In addition, it is assumed that mild liver fibrosis was observed in the liver at the time of the CT examination, but the patient had no subjective symptoms and no liver cirrhosis had developed and thus examination and analysis of liver fibrosis were not ordered. In addition, a situation in which liver fibrosis of the patient is getting worse day by day is assumed.

As shown in FIG. 2, the patient is monitored for diabetes regularly (daily to monthly). For example, the patient sends monitoring data measured by himself/herself using the monitoring device 3 to the medical information processing device 1 every day and receives information on a diabetes index value (first index value) from the medical information processing device 1 via the terminal device 5 or the like. This monitoring is continuously performed because special treatment is not required as long as the diabetes index value does not indicate an abnormality (risk “low”). The degree of progress of liver fibrosis is defined, for example, by an index in five stages from F0 to F4 (indicating that fibrosis is progressing from F0 to F4), and it is assumed that the degree of progress of liver fibrosis of the patient is maintained at F1.

Meanwhile, with the passage of time, liver fibrosis analysis processing is performed for the first time at the timing when the diabetes index value indicates an abnormal tendency (risk “medium”). In the liver fibrosis analysis processing, analysis based on past CT images of the patient stored in the diagnostic information database DB is performed. As a result of this analysis, treatment of controlling the progress of liver fibrosis (preventing or delaying the progression of liver fibrosis) is performed on the patient if progress of liver fibrosis has been observed (for example, if the degree of liver fibrosis is F2). Thereafter, diagnosis of diabetes is started at the timing when the diabetes index value indicates an abnormality (risk “high”) with the passage of time. As a result of this diagnosis, if diabetes is confirmed, treatment for diabetes is performed on the patient. A treatment method (e.g., a treatment method using tolbutamide) that is concerned about lowering liver function as a result of controlling liver fibrosis prior to diabetes treatment to prevent the progress of liver fibrosis can also be used to treat the diabetes. That is, it is possible to increase the likelihood of applying a better treatment method to diabetes by treating liver fibrosis at a stage before diabetes worsens and requires treatment.

Next, a processing flow of the medical information processing device 1 under conditions assumed in FIG. 2 will be described using FIG. 3. The flowchart shown in FIG. 3 is performed at a predetermined timing (for example, once a day) on the basis of monitoring conditions and the like. First, the first acquisition function 101 of the medical information processing device 1 acquires monitoring data regarding diabetes of the patient who is a target for diagnosis from the monitoring device 3 or the terminal device 5 via the communication network NW (step S101).

Next, the index value calculation function 103 calculates a diabetes risk index value of the patient who is a target for diagnosis on the basis of the monitoring data regarding diabetes acquired by the first acquisition function 101 (step S103).

Next, the determination function 104 determines whether or not analysis of liver fibrosis has already been performed for the patient who is a target for diagnosis (step S105). The determination function 104 determines whether or not analysis of liver fibrosis has already been performed, for example, on the basis of whether or not analysis results of liver fibrosis are stored in the diagnostic information database DB. If it is determined that liver fibrosis has already been analyzed (YES in step S105), processing of this flowchart ends.

On the other hand, if it is determined that analysis of liver fibrosis has not been performed (NO in step S105), the determination function 104 determines whether or not to perform analysis of liver fibrosis on the basis of a comparison between the diabetes risk index value and the threshold value (step S107). The determination function 104 determines whether or not to perform analysis of liver fibrosis, for example, on the basis of whether or not the diabetes risk index value is equal to or greater than the threshold value. For example, if the diabetes risk index value is “low,” analysis of liver fibrosis is not performed. On the other hand, for example, if the diabetes risk index value is “medium” or “high,” analysis of liver fibrosis is performed. If it is determined that analysis of liver fibrosis is not performed (NO in step S107), processing of this flowchart ends.

On the other hand, if it is determined that analysis of liver fibrosis is performed (YES in step S107), the second acquisition function 102 acquires diagnostic information (CT image) of the patient who is a target for diagnosis from the diagnostic information database DB via the communication network NW (step S109).

Next, the output function 105 outputs the diagnostic information (CT image) of the patient who is a target for diagnosis acquired by the second acquisition function 102 to the analysis device 7 (step S111). Accordingly, the analysis device 7 starts automatic analysis of a liver region using the diagnostic information acquired from the output function 105 and notifies the patient or a person related thereto (doctor or the like) of analysis results. The patient notified of the presence of liver fibrosis seeks medical attention at a medical institution and starts treatment for liver fibrosis. Accordingly, processing of this flowchart ends.

According to the first embodiment described above, it is possible to improve the accuracy of analysis and diagnosis by the analysis device 7 by narrowing down the number of targets for diagnosis for the second disease. In addition, it is possible to perform treatment for the second disease at a stage before the first disease worsens and requires treatment to increase the likelihood of applying a better treatment method to the first disease. For example, when the risk index value of the first disease (e.g., diabetes) becomes equal to or greater than a threshold value (e.g., “medium”), analysis of the second disease (e.g., liver fibrosis) on the basis of diagnostic information (e.g., CT image) on the patient captured in the past is performed. As a result, if the patient has developed the second disease at the time of undergoing a CT examination in the past, the presence of the second disease is noticed before treatment for the first disease is started, and thus treatment for the second disease can be started early. Some medicine for treatment of the first disease affect the function of the second disease (e.g., liver function), and some are contraindicated in patients with severe liver diseases (including advanced liver fibrosis, cirrhosis, and the like) and impaired liver function. Therefore, if treatment for the second disease can be started before treatment for the first disease becomes necessary, it is possible to consider applying a treatment method for the first disease which affects other functions and broaden the range of treatment. As a result, the patient will be able to proceed with the treatment for the first disease using an optimal treatment method. Treatment (liver fibrosis control) for the second disease (liver fibrosis) includes exercise guidance and dietary guidance. These treatment methods are also effective in preventing the first disease (diabetes). As a result, it is possible to expect secondary effects such as reducing the likelihood of the patient developing diabetes and the risk of diabetes becoming more severe.

In the first embodiment, it is assumed that analysis of the second disease (e.g., liver fibrosis) is automatically performed on the basis of diagnostic information acquired in the past. The patient does not need to undergo new diagnosis (CT imaging and the like) for diagnosis of liver fibrosis. In addition, since analysis is automatically performed by the analysis device 7, there is no need for a doctor to order an examination or to select an image. On the other hand, it is conceivable that a degree of progress of liver fibrosis at the time of CT examination of organs around the liver is different from a degree of progress of liver fibrosis at the time when the diabetes risk index value has become “medium.” Therefore, diagnosis results may be adjusted by adjusting parameters such as weights at the time of performing analysis of liver fibrosis by the analysis device 7 according to the degree of progress of liver fibrosis at the time of CT examination of organs around the liver and time intervals at the time when the diabetes risk index value has become “medium.”

### Second Embodiment

A second embodiment will be described below. A difference from the above-described first embodiment is that processing is performed under the condition that there is no diagnostic information (e.g., CT image) that has been acquired in the past with respect to a patient who is a target for diagnosis. In the following, differences from the first embodiment will be mainly described and descriptions of common points with the first embodiment will be omitted. In the description of the second embodiment, the same parts as in the first embodiment are assigned the same reference numerals.

### Processing Flow

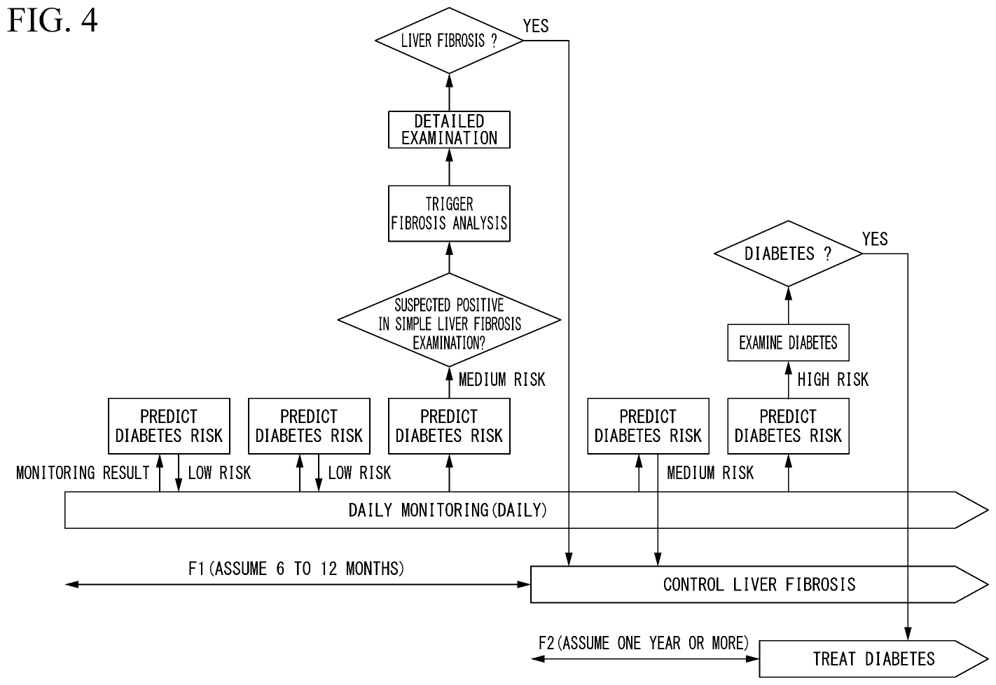

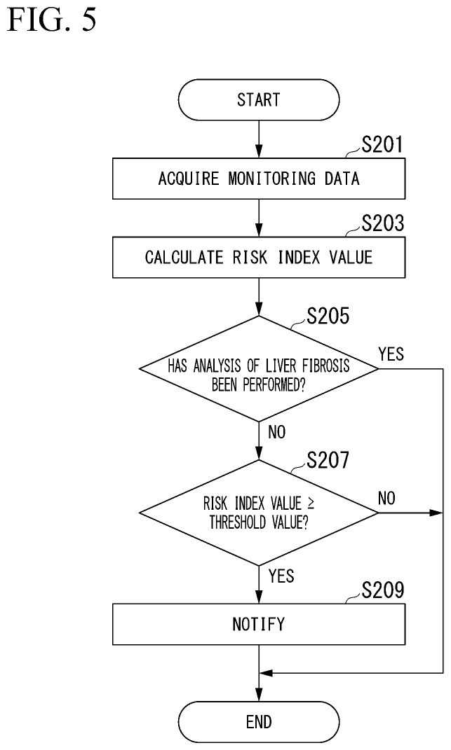

An example of a flow of processing of the medical information processing device 1 according to the second embodiment will be described. FIG. 4 is a diagram illustrating an example of a case in which the medical information processing device 1 according to the second embodiment is applied to a diagnosis flow. FIG. 5 is a flowchart showing an example of a flow of processing of the medical information processing device 1 according to the second embodiment. As in the description of the processing flow in the first embodiment, an example of a case in which the first disease is “diabetes” and the second disease is “liver fibrosis” will be described below. It is assumed that a patient who is a target for diagnosis has not undergone examination of organs around the liver (for example, examination of pneumonia) within the past one year and diagnostic information is not stored in the diagnostic information database DB. Alternatively, it is assumed that diagnostic information is stored in the diagnostic information database DB, but the stored diagnostic information does not match input conditions of the analysis device 7 and cannot be used for analysis.

As shown in FIG. 4, the patient is monitored for diabetes regularly (daily to monthly). This monitoring is only continuously performed because special treatment is not required as long as a diabetes index value does not indicate an abnormality (risk “low”). Then, with the passage of time, processing of analyzing liver fibrosis is performed for the first time at the timing when the diabetes index value indicates an abnormal tendency (risk “medium”). In this processing of analyzing for liver fibrosis, past CT images of the patient and the like are not stored in the diagnostic information database DB and thus it is necessary to acquire new CT images. For this reason, the patient or a person related thereto (here, the attending doctor of the patient) is notified that a liver fibrosis examination is required. The patient that has received the notification undergoes, for example, a simple liver fibrosis examination, and if the simple examination is positive, a detailed liver fibrosis examination is performed. Alternatively, the attending doctor who has received the notification recommends a liver fibrosis examination to the patient or orders a liver fibrosis examination, and the patient undergoes a simple liver fibrosis examination and a detailed examination according to an instruction of the attending doctor. The analysis device 7 analyzes for liver fibrosis examination data obtained by the examination and notifies the patient or the like of analysis results.

As a result of the analysis, if progress of liver fibrosis is observed (for example, if the degree of liver fibrosis is F2), treatment of controlling the progress of liver fibrosis (preventing the progress of liver fibrosis) is performed on the patient. Thereafter, diagnosis of diabetes is started at the timing when the diabetes index value indicates an abnormality (risk “high”) with the passage of time. As a result of this diagnosis, if diabetes is confirmed, treatment for diabetes is performed on the patient. A treatment method (e.g., a treatment method using tolbutamide) that is concerned about lowering liver function as a result of controlling liver fibrosis prior to diabetes treatment to prevent the progress of liver fibrosis can also be used to treat the diabetes.

Next, the processing flow of the medical information processing device 1 under the conditions assumed in FIG. 4 will be described using FIG. 5. The flowchart shown in FIG. 5 is performed at a predetermined timing (for example, once a day) on the basis of monitoring conditions and the like. First, the first acquisition function 101 of the medical information processing device 1 acquires monitoring data regarding diabetes from the monitoring device 3 or the terminal device 5 via the communication network NW (step S201).

Next, the index value calculation function 103 calculates a diabetes risk index value on the basis of the monitoring data regarding diabetes acquired by the first acquisition function 101 (step S203).

Next, the determination function 104 determines whether or not analysis of liver fibrosis has already been performed for the patient who is a target for diagnosis (step S205). If it is determined that analysis of liver fibrosis has already been performed (YES in step S205), processing of this flowchart ends.

On the other hand, if it is determined that analysis of liver fibrosis has not been performed (NO in step S205), the determination function 104 determines whether or not to perform analysis of liver fibrosis on the basis of a comparison between the diabetes risk index value and the threshold value (step S207). If it is determined that analysis of liver fibrosis is not performed (NO in step S207), processing of this flowchart ends.

On the other hand, if it is determined that analysis of liver fibrosis is performed (YES in step S207), the notification function 107 notifies the terminal device 5, a smart watch, or the like carried by the patient that a liver fibrosis examination is necessary via the communication network NW (step S209). In addition to or instead of notifying the patient, the notification function 107 notifies a doctor or the like by causing the display 130 to display that a liver fibrosis examination is necessary under the control of the display control function 106. For example, the patient that has received the notification undergoes a liver fibrosis examination. The analysis device 7 analyzes for liver fibrosis examination data obtained by the examination and notifies the patient or the like of analysis results. Thereafter, processing of controlling the progress of liver fibrosis is performed on the patient as needed. Accordingly, processing of this flowchart ends.

According to the second embodiment described above, it is possible to improve the accuracy of analysis and diagnosis by the analysis device 7 by narrowing down the number of targets for diagnosis for the second disease. In addition, it is possible to increase the likelihood of applying a better treatment method to the first disease by treating the second disease at a stage before the first disease worsens and requires treatment. Furthermore, since an examination ordered in response to a notification from the medical information processing device 1 is an examination for diagnosing the second disease (for example, liver fibrosis), it is possible to improve the accuracy of analysis of the second disease more than secondarily using diagnostic information imaged for the purpose of diagnosing another disease as in the first embodiment.

### MODIFIED EXAMPLE

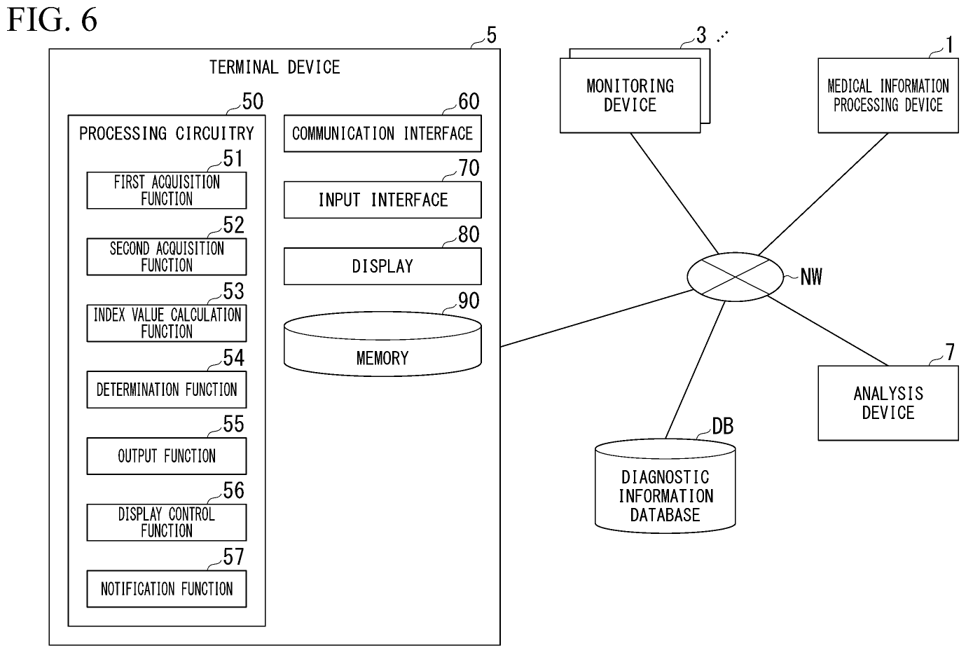

FIG. 6 is a diagram showing an example of a usage environment and functional blocks of a terminal device 5 according to a modified example. As shown in FIG. 6, only a difference from the first and second embodiments described above is that each function of the processing circuitry 100 of the medical information processing device 1 is realized in the terminal device 5 carried by the patient. By adopting such a configuration, it is possible to perform diagnostic processing only with equipment in the patient's home.

According to at least one embodiment described above, it is possible to contribute to improvement of the accuracy of diagnosis by including the first acquirer that acquires the first risk information regarding the first disease of the subject, and the second acquirer that acquires the second risk information regarding the second disease different from the first disease of the subject when the acquired first risk information satisfies a predetermined condition.

The embodiments described above can be represented as follows.

A medical information processing device including processing circuitry,
wherein the processing circuitry is configured to:
acquire first risk information regarding a first disease of a subject; and
acquire second risk information regarding a second disease different from the first disease of the subject when the acquired first risk information satisfies a predetermined condition.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device comprising processing circuitry configured to:
acquire first risk information regarding a first disease of a subject; and
acquire second risk information regarding a second disease different from the first disease when the acquired first risk information satisfies a condition based on a second threshold value which is less than a first threshold value related to treatment determination of the first disease and is greater than a normal range.

2. The medical information processing device according to claim 1, wherein the processing circuitry is further configured to determine to analyze the second disease when the acquired first risk information is equal to or greater than the second threshold value, or is greater than the second threshold value.

3. The medical information processing device according to claim 2, wherein the processing circuitry is further configured to:
output instruction information for instructing analysis of the second disease using diagnostic information on the subject to an external analysis device when determining that the analysis of the second disease is to be performed; and
acquire the second risk information, which is a result of the analysis of the second disease, from the external analysis device.

4. The medical information processing device according to claim 1, wherein the processing circuitry is further configured to acquire past diagnostic information on the subject and acquire the second risk information obtained by analyzing the past diagnostic information.

5. The medical information processing device according to claim 1, wherein the processing circuitry is further configured to notify of support information regarding the first or second disease on the basis of the acquired first or second risk information.

6. The medical information processing device according to claim 5, wherein the processing circuitry is further configured to notify of the support information for instructing treatment for the second disease to be started on the basis of the acquired second risk information.

7. The medical information processing device according to claim 1, wherein the processing circuitry is further configured to:
acquire monitoring data obtained by monitoring the subject; and
calculate the first risk information, which is an index value related to the first disease, on the basis of the acquired monitoring data.

8. The medical information processing device according to claim 1, wherein the second disease is a disease which inhibits treatment of the first disease.

9. A medical information processing method, using a computer of a medical information processing device, comprising:
acquiring first risk information regarding a first disease of a subject; and
acquiring second risk information regarding a second disease different from the first disease when the acquired first risk information satisfies a condition based on a second threshold value which is less than a first threshold value related to treatment determination of the first disease and is greater than a normal range.

10. A computer-readable non-transitory storage medium storing a program causing a computer of a medical information processing device to:
acquire first risk information regarding a first disease of a subject; and
acquire second risk information regarding a second disease different from the first disease when the acquired first risk information satisfies a condition based on a second threshold value which is less than a first threshold value related to treatment determination of the first disease and is greater than a normal range.
